(19) United States
(12) Patent Application Publication
(10) Pub. No.: US 2022/0087974 A1
(43) Pub. Date: Mar. 24, 2022

(54) METHOD AGAINST CORONAVIRUS INFECTION WITH COUMARIN-DERIVED COMPOUND

(71) Applicants: CHANG GUNG UNIVERSITY, Taoyuan City (TW); NATIONAL DEFENSE MEDICAL CENTER, Taipei City (TW)

(72) Inventors: Jim-Tong HORNG, New Taipei City (TW); Wen-Fang TANG, Taoyuan City (TW); Hui-Ping TSAI, Taipei City (TW); Chung-Fan HSIEH, Taoyuan City (TW); Chia-Yi LIN, Taoyuan City (TW); Guan-Hua LIN, Taoyuan City (TW); Yu-Li CHEN, Taoyuan City (TW); Po-Shiuan HSIEH, Taipei City (TW); Tien-Yao CHANG, Taipei City (TW); Yu-Hsiu CHANG, Taipei City (TW)

(21) Appl. No.: 17/333,266

(22) Filed: May 28, 2021

(30) Foreign Application Priority Data
Sep. 21, 2020 (TW) 109132585

(51) Int. Cl. A61K 31/37; A61P 31/14

(52) U.S. Cl. CPC A61K 31/37; A61P 31/14

(57) ABSTRACT

Disclosed herein is a method against coronavirus infection, which includes administering to a subject in need thereof an effective amount of 8-benzoyl-4-methyl-9-phenyl-furo[2,3-h]chromen-2-one or a pharmaceutically acceptable salt thereof.

This application claims priority to TW Patent Application No. 109132585 filed Sep. 21, 2020, the entire contents of which are hereby incorporated by reference.

### REFERENCE TO AN ELECTRONIC SEQUENCE LISTING

The contents of the electronic sequence listing (176-336 Sequence listing.txt; Size: 1,204 bytes; and Date of Creation: Jun. 23, 2021) is herein incorporated by reference in its entirety.

### FIELD

The present disclosure relates to a method against coronavirus infection with 8-benzoyl-4-methyl-9-phenyl-furo[2,3-h]chromen-2-one.

### BACKGROUND

8-benzoyl-4-methyl-9-phenyl-furo[2,3-h]chromen-2-one is a known coumarin-derived compound serving as a drug, which is represented by the following formula (I):

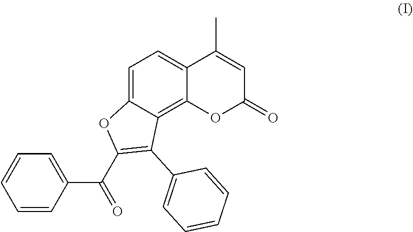 [SMILES: Cc1cc(=O)oc2c1ccc1oc(C(=O)c3ccccc3)c(-c3ccccc3)c12]

It has been reported that, 8-benzoyl-4-methyl-9-phenyl-furo[2,3-h]chromen-2-one can effectively inhibit viral replication during the early post infection stage, and can reduce the accumulation of viral genomes in host cells (Shin-Ru Shih et al. (2010), J. Antimicrob. Chemother., 65: 63-71). In addition, US Patent Application Publication No. 2012/0046238 A1 discloses that 8-benzoyl-4-methyl-9-phenyl-furo[2,3-h]chromen-2-one (i.e., Compound 1) exhibits antiviral activity against various viruses, such as Enterovirus 71 (EV71), Coxsackie virus B3, influenza viruses, human rhinovirus serotype 2 (HRV2), herpes simplex virus (HSV), hepatitis C virus (HCV), hepatitis B virus (HBV), Epstein-Barr virus (EBV), and human immunodeficiency virus (HIV).

Coronaviruses are a group of related RNA viruses that infect a variety of animal species including humans, such as severe acute respiratory syndrome coronavirus (SARS-CoV), middle east respiratory syndrome coronavirus (MERS-CoV), and human coronavirus 229E (HcoV-229E). Coronavirus disease 2019 (COVID-19) is an infectious disease caused by severe acute respiratory syndrome coronavirus 2 (SARS-CoV-2) recently discovered as a new coronavirus. Major symptoms include respiratory symptoms such as fever above 38° C., cough, shortness of breath, and difficulty in breathing. Symptoms such as loss of smell and taste, diarrhea, headache, chills, loss of appetite, general malaise, and impaired consciousness may be observed. At present, an effective curative treatment for COVID-19 has not been established, and symptomatic treatment is the center.

### SUMMARY

Therefore, an object of the present disclosure is to provide a method against coronavirus infection that can alleviate at least one of the drawbacks of the prior art.

The method includes administering to a subject in need thereof an effective amount of 8-benzoyl-4-methyl-9-phenyl-furo[2,3-h]chromen-2-one or a pharmaceutically acceptable salt thereof.

### BRIEF DESCRIPTION OF THE DRAWINGS

The above and other objects, features and advantages of the present disclosure will become apparent with reference to the following detailed description and the exemplary embodiments taken in conjunction with the accompanying drawings, in which:

FIG. 1 shows the cell viability rate in each group of Example 1, infra;

FIG. 2 shows the distribution profile of viral plaques in each group described in section A of Example 2, infra;

FIG. 3 shows the number of viral plaques in each group described in section A of Example 2, infra; and

FIG. 4 shows the relative RNA expression level of each group described in section B of Example 2, infra.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method against coronavirus infection, which includes administering to a subject in need thereof an effective amount of 8-benzoyl-4-methyl-9-phenyl-furo[2,3-h]chromen-2-one (which is referred to as “HP520-2” hereinafter) or a pharmaceutically acceptable salt thereof.

As used herein, the term “against coronavirus infection” or “anti-coronavirus infection” means prevention of infection by a coronavirus, suppression of coronavirus replication, and/or treatment and/or prevention of infectious diseases caused by a coronavirus.

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

As used herein, the term “pharmaceutically acceptable salt” refers to any salt, which, upon administration to the subject is capable of providing (directly or indirectly) a compound as described herein (i.e., HP520-2) without undue toxicity, irritation, allergic response and the like. In particular, “pharmaceutically acceptable salt” may encompass those approved by a regulatory agency of the federal or a state government or listed in the U.S. Pharmacopeia or other generally recognized pharmacopeia for use in animals, and more particularly in humans. The preparation of salts can be carried out by methods known in the art.

For instance, the pharmaceutically acceptable salts of HP520-2 may be acid addition salts, base addition salts or metallic salts, and they can be synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture thereof. Examples of the acid addition salts may include mineral acid addition salts such as, for example, hydrochloride, hydrobromide, hydroiodide, sulfate, nitrate, and phosphate; and organic acid addition salts such as, for example, acetate, maleate, fumarate, citrate, oxalate, succinate, tartrate, malate, mandelate, methanesulphonate, p-toluenesulphonate, 2-naphtalenesulphonate, and 1,2-ethanedisulphonate. Examples of the alkali addition salts may include inorganic salts such as, for example, ammonium; and organic alkali salts such as, for example, ethylenediamine, ethanolamine, N,N-dialkylenethanolamine, triethanolamine, choline, glucamine, and basic amino acids salts. Examples of the metallic salts may include, for example, sodium, potassium, calcium, magnesium, aluminium, and lithium salts.

According to the present disclosure, the coronavirus infection may be caused by a coronavirus selected from the group consisting of severe acute respiratory syndrome coronavirus (SARS-CoV), severe acute respiratory syndrome coronavirus 2 (SARS-CoV-2), middle east respiratory syndrome coronavirus (MERS-CoV), human coronavirus 229E (HcoV-229E), and combinations thereof.

According to the present disclosure, HP520-2 or the pharmaceutically acceptable salt thereof may be prepared into a pharmaceutical composition in a dosage form suitable for, e.g., parenteral or oral administration, using technology well known to those skilled in the art. The suitable dosage form includes, but is not limited to, injections (e.g., sterile aqueous solutions or dispersions), sterile powder, tablets, troches, lozenges, capsules, dispersible powder, granule, solutions, suspensions, emulsions, syrup, elixirs, slurry, and the like.

According to the present disclosure, the pharmaceutical composition may be administered by parenteral routes selected from the group consisting of intraperitoneal injection, intrapleural injection, intramuscular injection, intravenous injection, intraarterial injection, intraarticular injection, intrasynovial injection, intrathecal injection, intracranial injection and sublingual administration.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, fillers, wetting agents, lubricants, absorption delaying agents, liposomes, and the like. The choice and amount of the aforesaid agents are within the expertise and routine skills of those skilled in the art.

According to the present disclosure, the dosage and the frequency of administration of the pharmaceutical composition may vary depending on the following factors: the severity of the disease to be treated, the route of administration, and the weight, age, physical condition and response of the subject to be treated. The daily dosage of the pharmaceutical composition may be administered in a single dose or in several doses.

According to the present disclosure, the pharmaceutical composition containing HP520-2 may be formulated into an external preparation (such as a hand sanitizer or a hand washing agent) suitable for application to the hands or skin using technology well known to those skilled in the art. The external preparation includes, but is not limited to, an emulsion, a soap, a gel, an ointment, a cream, an aerosol, a spray, a lotion, a serum, a paste, a foam, and a drop.

According to the present disclosure, the pharmaceutical composition containing HP520-2 is easy to apply, low in toxicity, environmentally friendly, and not bioaccumulative, and thus can be used as an environmental disinfectant (such as a surface cleaner, a detergent, and a sterilant).

According to the present disclosure, the pharmaceutical composition may further include remdesivir serving as a synergistic antiviral agent.

The disclosure will be further described by way of the following examples. However, it should be understood that the following examples are solely intended for the purpose of illustration and should not be construed as limiting the disclosure in practice.

### Examples

### General Experimental Materials:

### 1. Source and Cultivation of Vero E6 Cells

African green monkey kidney (Vero E6) cells were obtained from the Chang Gung Medical Foundation, the Linkou Chang Gung Memorial Hospital (Taiwan). The Vero E6 cells were grown in a 10-cm Petri dish containing Dulbecco's Modified Eagle's Medium (DMEM) (Cat. No. 12000-061, Gibco) supplemented with 10% fetal bovine serum (FBS) (Cat. No. 26140-079, Gibco), which is referred to as “E10 medium” hereinafter. The Vero E6 cells were cultivated in an incubator with culture conditions set at 37° C. and 5% CO₂. Medium change was performed every two to three days. Cell passage was performed when the cultured cells reached 80%-90% of confluence.

### 2. Virus Strains

Severe acute respiratory syndrome coronavirus 2 (SARS-CoV-2) and human coronavirus 229E (HcoV-229E) used in the following experiments were provided by the Chang Gung Medical Foundation, the Linkou Chang Gung Memorial Hospital (Taiwan).

A respective one of SARS-CoV-2 and HcoV-229E was dissolved in DMEM (Cat. No. 12000-061, Gibco) supplemented with 2% FBS (Cat. No. 26140-079, Gibco), which is referred to as “E2 medium” hereinafter, so as to prepare a SARS-CoV-2 solution having a virus amount of 5.73×10⁶ pfu/mL and a HcoV-229E solution having a virus amount of 6.6×10⁶ pfu/mL. The two virus solutions were stored in a freezer at −80° C. for further experiment.

### Example 1. Evaluation for the Effect of 8-benzoyl-4-methyl-9-phenyl-furo[2,3-h]chromen-2-one Against HCoV-229E

### Experimental Procedures:

Vero E6 cells were divided into 5 groups, including one normal control group, one pathological control group, and three experimental groups (i.e., experimental groups 1 to 3). Each group of the Vero E6 cells was incubated in a respective well of a 96-well culture plate containing 100 μL of E2 medium at 2×10⁴ cells/well, followed by cultivation in an incubator (37° C., 5% CO₂) for 24 hours. Afterwards, the culture medium in each well was removed, and the cells of the experimental groups 1 to 3 were pretreated with 8-benzoyl-4-methyl-9-phenyl-furo[2,3-h]chromen-2-one (i.e., HP520-2) (Cat. No. STL-513320, Vitas-M Laboratory) respectively at concentrations of 20 nM, 40 nM, and 80 nM, followed by adding 150 μL of the HcoV-229E solution prepared in section 2 of General Experimental Materials.

In addition, the cells of the pathological control group were added with 50 μL of E2 medium, followed by adding 150 μL of the HcoV-229E solution prepared in section 2 of General Experimental Materials. The cells of the normal control group were added with 200 μL of E2 medium, and were not treated with the HcoV-229E solution prepared in section 2 of General Experimental Materials.

Each group was cultivated in an incubator (37° C., 5% CO₂) for 96 hours. The liquid in each well was removed, followed by adding 50 μL of 3-[4,5-dimethylthiazol-2-yl]-2,5-diphenyltetrazolium bromide (MTT). After cultivation in an incubator (37° C., 5% CO₂) for 2 hours, the respective resultant cell culture was added with 150 μL of dimethyl sulfoxide (DMSO), followed by subjecting the mixture thus obtained to determination of absorbance at a wavelength of 590 nm (OD₅₉₀) by an ELISA reader.

The cell viability rate (%) was calculated using the following Equation (I):

A=(B/C)×100 (I)

where A=cell viability rate (%)

B=OD₅₉₀ value of respective group

C=OD₅₉₀ value of normal control group

In addition, the 50% effective concentration (EC₅₀) was determined from the linear portion of the plotted dose-response curve by calculating the concentration of active ingredient that reduced absorbance in the treated cells, as compared to the pathological control cells, by 50% (n=3). The experimental data are expressed as mean±SD (standard deviation).

### Results:

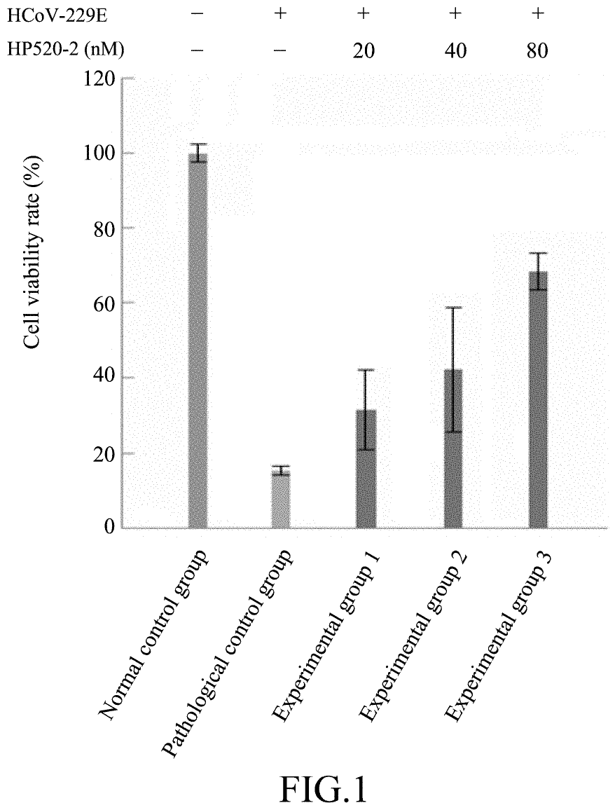

FIG. 1 shows the cell viability rate of each group. It can be seen from FIG. 1 that the cell viability rates determined in the experimental groups 1 to 3 were significantly higher than that determined in the pathological control group, and HP520-2 exhibited dose-related antiviral effect. In addition, the EC₅₀ value of HP520-2 is 39±15 nM.

Summarizing the test results above, it is clear that HP520-2 can act effectively against HcoV-229E infection.

### Example 2. Evaluation for the Effect of HP520-2 Against SARS-CoV-2

### A. Viral Plaque Reduction Assay:

Vero E6 cells were divided into 2 groups, including one pathological control group and one experimental group. Each group of the Vero E6 cells was incubated in a respective well of a 24-well culture plate containing 0.5 mL of E2 medium at 4×10⁵ cells/well, followed by cultivation in an incubator (37° C., 5% CO₂) for 24 hours. Afterwards, the culture medium in each well was removed, and the cells of each group were infected with SARS-CoV-2 at a multiplicity of infection (m.o.i.) of 0.01. After cultivation in an incubator (37° C., 5% CO₂) for 1 hour, the liquid in each well was removed, and the SARS-CoV-2-infected Vero E6 cells of each group were washed twice with phosphate-buffered saline (PBS).

Thereafter, the SARS-CoV-2-infected Vero E6 cells of the experimental group were overlaid with E2 medium containing 1.4% methyl cellulose and 1 FM HP520-2, and the SARS-CoV-2-infected Vero E6 cells of the pathological control group were overlaid with E2 medium containing 1.4% methyl cellulose.

After cultivation in an incubator (37° C., 5% CO₂) for 72 hours, the cells of each group were fixed with 0.5 mL of a 4% paraformaldehyde solution at room temperature for 1 hour. Afterwards, the fixed cells in each well were stained with 1% crystal violet for 20 minutes. After rinsing the stained cells with water, distribution of the viral plaques in each well was analyzed by visual observation, and the number of viral plaques of each group was counted.

### Results:

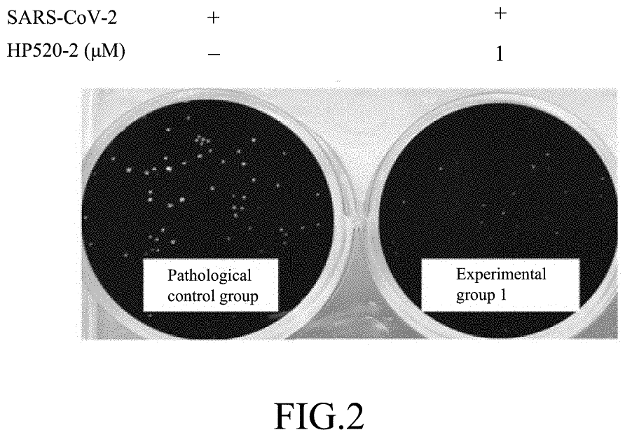

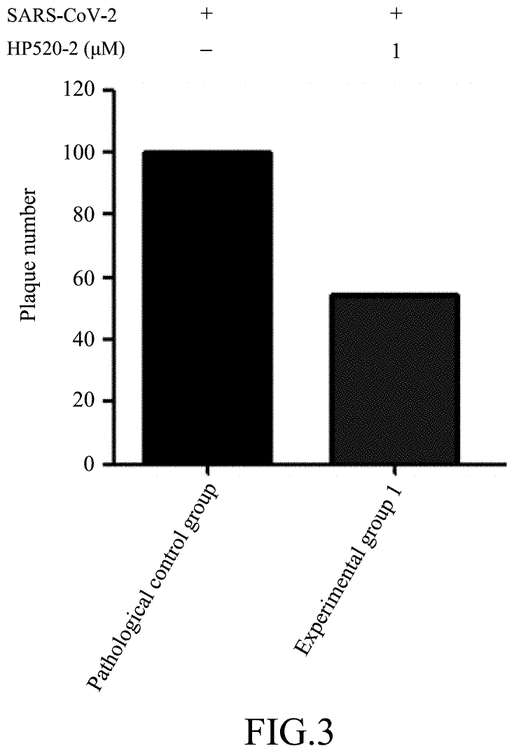

Referring to FIGS. 2 to 3, the number of viral plaques determined in the experimental group was lower than that determined in the pathological control group, indicating that HP520-2 is effective in reducing viral replication in host cells infected with SARS-CoV-2.

### B. Quantitative Determination of Viral Gene Expression:

Vero E6 cells were divided into 4 groups, including one pathological control group and three experimental groups (i.e., experimental groups 1 to 3). Each group of the Vero E6 cells was incubated in a respective well of a 24-well culture plate containing 100 μL of E10 medium at 4×10⁵ cells/well, followed by cultivation in an incubator (37° C., 5% CO₂) for 24 hours. Afterwards, the culture medium in each well was removed, and the cells of the experimental groups 1 to 3 were pretreated with HP520-2 respectively at concentrations of 3.906 nM, 15.625 nM, and 62.5 nM, followed by being treated with SARS-CoV-2 at a m.o.i. of 0.01.

In addition, the cells of the pathological control group were treated with SARS-CoV-2 at a m.o.i. of 0.01, and were not treated with HP520-2.

Each group was cultivated in an incubator (37° C., 5% CO₂) for 1 hour. The liquid in each well was collected, and was subjected to total RNA extraction using TRIzol reagent (Invitrogen, Thermo Fisher Scientific, Carlsbad, Calif.) in accordance with the manufacturer's instructions.

Thereafter, 1 μg of the resultant RNA of the respective group was used as a template for synthesizing cDNA by reverse transcription polymerase chain reaction (RT-PCR) using M-MLV reverse transcriptase (Invitrogen, USA). The thus obtained cDNA, serving as a DNA template, was diluted 100-fold with deionized distilled H₂O, and was subjected to real-time PCR, which was performed on a StepOnePlus real-time PCR system (Applied Biosystems) using TaqMan™ real-time kit and the reaction conditions shown in Table 1, so as to determine the changes in Envelope (E) gene expression. Glyceraldehyde-3-phosphate dehydrogenase (GAPDH) gene was used as an endogenous control in the quantitative analysis of real-time PCR to normalize the gene expression data.

The SARS-CoV-2-E gene-specific primer set and the GAPDH gene-specific primer set were purchased from TaqMan (Thermo Fisher Scientific). The detailed information of the abovementioned primer pairs is summarized in Table 2.

TABLE 1

| | | Volume |
|---|---|---|
| Reaction mix | | (μL) |
| cDNA | | 5 |
| SARS-CoV-2-E | Forward primer (100 nM) | 1 |
| gene-specific primer | Reverse primer (100 nM) | 1 |
| set | | |
| GAPDH gene-specific | Forward primer (100 nM) | 1 |
| primer set | Reverse primer (100 nM) | 1 |

| | |
|---|---|
| Smart Quant Green Master Mix with dUTP Low ROX | 10 |
| mixture (Protech Technology, Taipei, Taiwan) | |
| Diethyl pyrocarbonate (DEPC)-treated water | 3 |

Operation conditions: forty cycles of the following
reactions: denaturation at 95° C. for 10 minutes, annealing
at 95° C. for 15 seconds, and extension at 60° C. for 1 minute.

TABLE 2

| | | Nucleotide sequence | SEQ ID |
|---|---|---|---|
| Target gene | Primer | (5′→3′) | NO. |
| SARS-CoV-2-E | Forward | acaggtacgttaatagttaatagcgt | 1 |
| gene | primer | | |
| | Reverse | atattgcagcagtacgcacaca | 2 |
| | primer | | |
| GAPDH gene | Forward | tgcaccaccaactgcttagc | 3 |
| | primer | | |
| | Reverse | ggcatggactgtggtcatgag | 4 |
| | primer | | |

To quantify the changes in gene expression, the change in threshold cycle (ΔC_{T}) method was used to calculate the relative fold changes normalized against the GAPDH gene.

### Results:

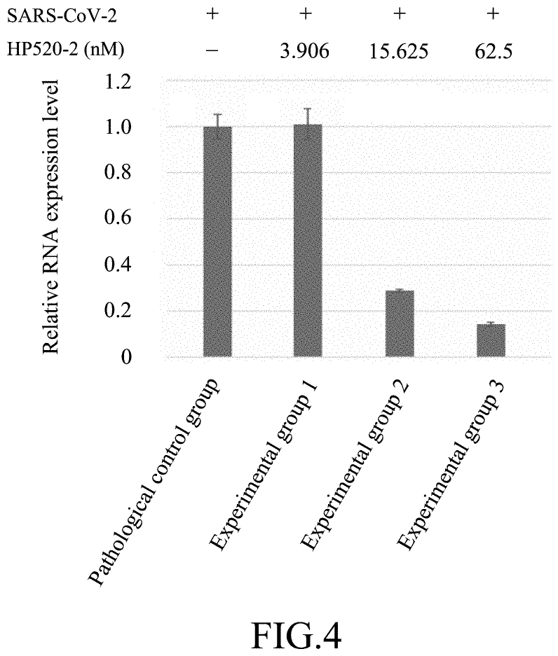

FIG. 4 shows the relative RNA expression level of E gene of SARS-CoV-2 in the infected Vero E6 cells pretreated with HP520-2. It can be seen from FIG. 4 that the RNA expression levels of the experimental groups 2 to 3 were significantly lower than that of the pathological control group, indicating that HP520-2 can reduce the viral gene expression and thereby inhibit the viral replication in the host cells.

All patents and references cited in this specification are incorporated herein in their entirety as reference. Where there is conflict, the descriptions in this case, including the definitions, shall prevail.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method against coronavirus infection, comprising administering to a subject in need thereof an effective amount of 8-benzoyl-4-methyl-9-phenyl-furo[2,3-h]chromen-2-one or a pharmaceutically acceptable salt thereof.

2. The method according to claim 1, wherein the coronavirus infection is caused by a coronavirus selected from the group consisting of severe acute respiratory syndrome coronavirus (SARS-CoV), severe acute respiratory syndrome coronavirus 2 (SARS-CoV-2), middle east respiratory syndrome coronavirus (MERS-CoV), human coronavirus 229E (HcoV-229E), and combinations thereof.

3. The method according to claim 1, wherein the 8-benzoyl-4-methyl-9-phenyl-furo[2,3-h]chromen-2-one or the pharmaceutically acceptable salt thereof is in a dosage form for oral administration.

4. The method according to claim 1, wherein the 8-benzoyl-4-methyl-9-phenyl-furo[2,3-h]chromen-2-one or the pharmaceutically acceptable salt thereof is in a dosage form for parenteral administration.


SEQUENCE LISTING (4 sequences)

SEQ ID NO: 1 - DNA, 26 bp, Artificial Sequence
  Forward primer for real-time PCR of SARS-CoV-2-E gene
acaggtacgttaatagttaatagcgt

SEQ ID NO: 2 - DNA, 22 bp, Artificial Sequence
  Reverse primer for real-time PCR of SARS-CoV-2-E gene
atattgcagcagtacgcacaca

SEQ ID NO: 3 - DNA, 20 bp, Artificial Sequence
  Forward primer for real-time PCR of GAPDH gene
tgcaccaccaactgcttagc

SEQ ID NO: 4 - DNA, 21 bp, Artificial Sequence
  Reverse primer for real-time PCR of GAPDH gene
ggcatggactgtggtcatgag